(19) United States
(12) Patent Application Publication
(10) Pub. No.: US 2016/0136208 A1
(43) Pub. Date: May 19, 2016

(54) APPLICATION OF ADSORBED DRONE BROOD HOMOGENATE AND OF VITAMIN D3 OR D-GROUP VITAMINS AND/OR ACTIVE METABOLITES THEREOF FOR PREVENTING AND TREATING VIRAL DISEASES

(71) Applicant: OBSCHESTVO S OGRANICHENNOJ OTVETSTVENNOSTJU "PARAFARM", Penza (RU)

(72) Inventors: Villorij Ivanovich Strukov, Penza (RU); Mikhail Dmitrievich Prokhorov, Penza (RU); Olga Jones-Strukova, Grand Prairie, TX (US); Vjacheslav Nikolaevich Trifonov, Zarechnyj (RU); Julija Anatol'evna Elistratova, Penza (RU); Konstantin Gennad'evich Elistratov, Penza (RU); Natalya Vjacheslavovna Kurus', Penza (RU); Natalya Vyacheslavovna Eremina, Penza (RU); Marina Nikolaevna Maksimova, Penza (RU); Ramziya Timurshojvna Galeeva, Penza (RU); Larisa Grigorievna Radchenko, Penza (RU); Aleksandr Viktorovich Fedorov, Penzenskaya oblast', Kuznetsk (RU); Evgeny Nikolaevich Krutyakov, Penza (RU); Tatiana Viktorovna Elistratova, Penza (RU); Irina Vladimirovna Khomyakova, Penza (RU); Galina Anatolievna Tolbina, Penza (RU)

(73) Assignee: Obshchestvo s ogranichennoj otvetstvennost'ju <&ldquo;>Parafarm<&rdquo;>, Penza (RU)

(21) Appl. No.: 14/902,562

(22) PCT Filed: Jul. 3, 2014
(86) PCT No.: PCT/RU2014/000487

§ 371 Date: Jan. 1, 2016

(30) Foreign Application Priority Data
Jul. 3, 2013 (RU) 2013130302

(51) Int. Cl. A61K 35/64; A61K 31/593

(52) U.S. Cl. CPC A61K 35/64; A61K 31/593

(57) ABSTRACT

The invention relates to medicine, in particular, to agents for prophylaxis and treatment of conditions associated with various forms of catarrhal diseases, as well as for immunity improvement.

The invention relates to medicine, in particular, to agents for prophylaxis and treatment of conditions associated with various forms of catarrhal diseases, as well as for immunity improvement.

It is known that adsorbed drone brood homogenate and vitamin D3 or other D group vitamins (and/or their active metabolites) are used for closing cavity formations in metaphyseal (trabecular) bone sections, and for preventing secretion of calcium therefrom, which provides the reduction and elimination of bone cavities, and prevents the formation thereof (patent RU 92466732, 31 May 2011).

While conducting the studies of aforementioned “Osteo-vit D3” for the prophylaxis and treatment of bone tissue hypermineralization (during 2012-2013), it was found that there were no cases of respiratory viral diseases, particularly, influenza, in patients who received this preparation. Moreover, it was found that general well-being and immunity improved in patients receiving the aforementioned preparation.

During these studies no other drug having similar composition has been found, which allows the achievement the mentioned effect.

Hence, it was found that a new technical result, namely, the prophylaxis and treatment of catarrhal and respiratory viral diseases, is achieved due to the administration of the known drug (adsorbed drone brood homogenate and vitamin D3 or other D group vitamins and/or their active metabolites).

This technical result is achieved due to daily intake of 10 mg to 1000 mg of adsorbed drone brood homogenate and 50 ME to 100,000 ME of vitamin D3 or D group vitamins (and/or their active metabolites).

The wide dose range of the claimed drug is explained by individual characteristics of patient: age, diet, lifestyle, race, country of residence, gender, inherited and past illnesses. By evaluating these criteria, doctor selects the individual doses of components of the claimed drug and makes corrections based on the dynamics of treatment.

### EXAMPLE 1

The study on the efficiency of the preparation was conducted in a group, consisting of 22 patients aged from 32 to 68 years, among them 9 were male, 13—female. The patients received 1 tablet of the preparation 2 times a day during three-month course three times a year with monthly intervals by oral or sublingual administration in order to determine protective efficiency of the preparation against catarrhal and respiratory diseases.

As a result of the study, after 6-month therapy in the aforementioned group the immunity improvement was clinically observed, which was confirmed by objective examination data: there were no cases of acute respiratory disease or influenza in the patients during the autumn and winter period.

### EXAMPLE 2

Additional studies were conducted after the aforementioned studies. A group, consisting of 9 patients, aged from 21 to 59 years, 4 of them were male, 5—female, with the initial stage of influenza received 1 tablet of the aforementioned preparation two times a day during 1 month via sublingual administration.

As a result, the acute stage with the expressed symptoms of viral respiratory disease didn't develop in the patients of the studied group: the temperature, headache, chills and runny nose were absent. There were no such complications as bronchitis.

### EXAMPLE 3

Studies were conducted in a group of 11 females aged from 37 to 64 years with ARVI diagnosis. The preparation was prescribed to the aforementioned group in a dose of 1 tablet two times a day, during one month via sublingual administration.

As a result, in the patients of this group the disease was in a milder form: the body temperature didn't increase over 38 degrees, severe nasal congestion and runny nose were absent. Moreover, the recovery period was by 2-5 days shorter that in patients, who didn't receive the preparation.

We claim:

1. The use of 10 mg to 1000 mg of the adsorbed drone brood homogenate daily and 50 ME to 100,000 ME of vitamin D or D group vitamins (and/or their active metabolites) daily for prophylaxis and treatment of viral diseases.
